(19) United States
(12) Patent Application Publication
(10) Pub. No.: US 2023/0012462 A1
(43) Pub. Date: Jan. 12, 2023

(54) METHOD OF MAKING LARGE NEEDLE INSERTION INTO AN ARTERY OR VEIN EASIER AND WITH LESS UNNECESSARY TRAUMA TO THE BLOOD VESSEL AND METHODS FOR MANUFACTURING SAME (NISE)

(71) Applicant: HemoCleanse Technologies LLC, Lafayette, IN (US)

(72) Inventors: STEPHEN ASH, Lafayette, IN (US); THOMAS SULLIVAN, Lafayette, IN (US)

(73) Assignee: HemoCleanse Technologies LLC, Lafayette, IN (US)

(21) Appl. No.: 17/861,959

(22) Filed: Jul. 11, 2022

Related U.S. Application Data
(60) Provisional application No. 63/219,946, filed on Jul. 9, 2021.

(51) Int. Cl. A61M 25/06

(52) U.S. Cl. CPC A61M 25/0643; A61M 25/0693

(57) ABSTRACT

This disclosure relates to methods and exemplary devices for making needle insertion safer and easier (NISE). This NISE device includes at small needle, cannula, and large needle in a nested assembly to provide less trauma to a target vessel during large needle insertion. The small needle is movable relative to a body of the device and within the cannula to a position exterior the cannula. The cannula is movable within the large needle to a position exterior to the large needle. In a preferred assembly, the cannula is provided with a dulled tip and successive cutting edges along a tapered shape from the narrow dulled tip to the cannula body, such that the cannula blunt tip prevents trauma during insertion while the successive cutting edges generally enlarge the opening for placement of the large needle. The device can be provided in a stand alone device or alternately integrated into existing off the shelf and currently available vascular devices.

### CROSS REFERENCE TO RELATED APPLICATIONS

This U.S. Patent Application claims priority to U.S. Provisional Patent Application No. 63/219,946 filed 9 Jul. 2021 to the above-named inventors of which the disclosure is considered part of the disclosure of this application and is herein incorporated by reference in its entirety.

### FEDERALLY SPONSORED RESEARCH OR DEVELOPMENT

Not Applicable

### SEQUENCE LISTING, A TABLE, OR A COMPUTER PROGRAM

Not Applicable

### FIELD OF THE INVENTION

This invention relates generally to large needles and a large needle assembly and method for use and manufacturing. More particularly, embodiments of the invention provide a nested assembly of a small needle, cannula, and large needle in a coaxial aligned assembly for receipt in a support body. Further, the invention relates generally a large needle assembly, a method of manufacture and use for making large needle insertion into an artery or vein easier and with less unnecessary trauma to the blood vessel.

### BACKGROUND

It is often medically necessary to gain access to a vessel using a large needle, the outside diameter of which may be a substantial fraction of the diameter of the vessel. This requires accurate placement of the large needle to avoid unnecessary trauma to the vessel. Some vessels may have thick or tough walls making insertion difficult.

For insertion of a large needle or catheter into a vessel, the tip of the needle or catheter must be sharp to puncture the surrounding tissues and vessel wall. However, for stability and surety that the needle or catheter stays in the vessel and in the proper position, the needle or catheter must be long enough to be advanced into the vessel a distance much greater than the diameter of the vessel. This can be done with a needle, but with the tip of a needle being very sharp, advancing it in any direction may place the tip in contact with the vessel wall resulting in potential damage to the delicate vessel lining. Therefore, the person placing the needle must be able to sense when the tip enters the vessel and then lower the angle of insertion so that the needle tip stays central within the vessel while the needle is advanced. This takes considerable experience and skill but is still subject to chance and potential vessel damage due to normal variability in anatomy from patient to patient and in patients over time, change of medical condition.

One solution within the art to this problem, is the use of a blunt tipped plastic cannula with an internal sharp needle that is used to enter the vessel. This is the standard peripheral IV cannula. After the needle and catheter have entered the vessel, the needle can be retracted so that the blunt-tipped catheter is next advanced into the vessel. This works reasonably well but inserting the catheter portion requires more force than inserting the needle and deforms the vessel. Often the needle enters the vessel properly, but the catheter does not. The catheter will not then advance to its correct position within the vessel.

For the specific medical procedure of kidney dialysis, accesses are typically surgically arterialized vessels (fistulas) or plastic tubes (grafts) which have a very thick and tough wall, making the insertion of needles sometimes difficult. Large catheters with internal needles are even more difficult to insert, due to the lack of a cutting surface on the catheter. Accordingly, using what is called a fistula needle provides better blood flow at less pressure drop, because the needle walls are much thinner than the walls of plastic catheters. The catheters suited for fistulas and dialysis access are also considerably more expensive than fistula needles. For all these reasons, needles are inserted into fistulas and grafts routinely, not catheters. Often the points of these needles penetrate the other side of the accessed vessel, near to the entry site. Especially when the interior of the access is irregular in shape or has stenosis, it is difficult to advance the needle within the fistulas or grafts, because the tip impinges on the inside wall of the access. Invariably each point of contact of the needle point will cause damage to the delicate intimal layer of the interior surface of the accessed vessel.

Another solution to having a sharp entry point but then a blunt object for advancing within the vessel interior is the “Seldinger” technique. For this technique, a sharp needle is inserted in the vessel, and then a blunt “guidewire” is inserted through the needle and advanced into the vessel. The needle is then removed and the guidewire is advanced, usually under fluoroscopic imaging in the radiology department. A dilator with a surrounding catheter is advanced over the guidewire, and the dilator and guidewire are then removed, and the catheter is ready for use. Everything used in the technique must be sterile and protected from contamination. The person placing the device and assistants must don sterile gloves and gowns, and a wide sterile surgical field must be created around the insertion site to the vessel. The guidewires are usually long and flexible, so maintaining their sterility requires attention to the varying course of the guidewire. This technique works well in a radiology department but is not practical for dialysis units or stand-alone dialysis centers.

### BRIEF SUMMARY OF THE INVENTION

The present invention in the form of an improved large needle assembly and method for use, is generally referred to herein by the acronym N.I.S.E or NISE standing for: Needle Insertion Safer and Easier (“NISE”). The NISE device and method for use is intended to alleviate the disadvantages of the prior art by realizing at least three concepts: The first concept is the use of blunted points on penetrators other than the first penetrator, of which the first penetrator is retracted into a cylindrical cannula after initial vessel entry. The second is that the cannula has increasing diameters and cutting surfaces to enlarge the entry hole as it is advanced into the vessel. The third is that the access needle or catheter are carried into the vessel while surrounding the cannula. The cannula and the sharp needle (retracted into the cannula) are removed, leaving the access needle or catheter within the access, ready for use.

For better understanding, it may be best to first explain the method of use of the NISE device relative to the inventive structures and features, which will be discussed in an additional detail. In use of the device for the insertion of a large needle into a vessel, the first step is the entry of a small needle into the vessel, allowing the operator to verify entry into the vessel by blood emission or sight of blood at the NISE device. This small needle is sharp for easy initial entry. If this needle is wrongly inserted, for example, by hitting a vessel wall, trauma is minimal due to the small size of this first small needle.

Second, a larger cylindrical cannula, with the larger cylindrical cannula surrounding the small needle and coaxial with the small needle received within an aperture of the larger cylindrical cannula, is advanced with the needle a short distance into the target vessel. The position of the larger cylindrical cannula is held constant while the small needle is retracted (preferably done by spring action, controlled by the user) within a body of the device. The larger cylindrical cannula has a relatively dull tip and typically sharp outer cutting edges near or adjacent the tip. The large cylindrical cannula has an expanding size and cutting surfaces to enlarge the hole in the vessel as the large cylindrical cannula is advanced into the vessel. As the larger cylindrical cannula is advanced into the vessel, if contact is made with the opposite or an adjacent vessel wall, the dull tip of the large cylindrical cannula will minimize potential trauma to the vessel interior walls.

Third, when the large cylindrical cannula is fully advanced into the vessel, a large needle or catheter, referred to herein as an access needle and surrounding a posterior portion of the large cylindrical cannula, is brought into the vessel. If the large cylindrical cannula was advanced a full portion of a length of the large cylindrical cannula into the vessel, and the access needle is completely inserted in the vein, then the access needle is held in place and the cannula and enclosed small needle are removed from the device. If, however, the tip of the cannula met some obstruction during advancement in the vessel and the access needle was only partly inserted into the fistula, then the large cylindrical cannula can be held in place while the access needle is advanced over it.

Fourth, on removal of the large cylindrical cannula and small needle from the access needle interior, a tip of the small needle is drawn through an external tubing segment, elastomeric seal, or valve which automatically seals against leaks. The tubing which is attached to the access needle is then ready for blood removal or fluid infusion during the medical procedure.

In summary, the device includes three separate components which enter the vessel successively: the small sharp needle which first enters the vessel, the large cylindrical cannula having a tip surrounding the small sharp needle and having enlarging diameters and/cutting surfaces along its length (going outwards along the length from the cannula tip), and the large access needle surrounding the outer part of the large cylindrical cannula.

In operation, the sharp small needle is retracted into the large cylindrical cannula tip after insertion of both to the vessel. The large cylindrical cannula and access needle are then advanced into the vessel, and the cannula and small needle are then removed when the access needle is inserted to the proper position within the vessel.

These structures and their functions are all preferably contained in a single integrated unit, part of which may consist of slightly modified presently available vessel access products, Other features and capabilities may be added as described in the detailed description of the embodiments of this disclosure.

The invention now will be described more fully hereinafter with reference to the accompanying drawings, which are intended to be read in conjunction with both this summary, the detailed description and any preferred and/or particular embodiments specifically discussed or otherwise disclosed. This invention may, however, be embodied in many different forms and should not be construed as limited to the embodiments set forth herein; rather, these embodiments are provided by way of illustration only and so that this disclosure will be thorough, complete and will fully convey the full scope of the invention to those skilled in the art.

### BRIEF DESCRIPTION OF THE DRAWINGS

FIG. 1 shows a scheme of the basic stages of NISE device insertion from top to bottom relative to FIG. 1, according to the present disclosure;

FIG. 2 is a schematic top view of an exemplary NISE device, according to the present disclosure;

FIG. 3 is a perspective view of the top and right side view of a NISE device, with the small needle exposed, according to the present disclosure;

FIG. 4 is a perspective view of the bottom and left side view of the NISE device, with the small needle exposed, according to the present disclosure;

FIG. 5 is the right side view of the NISE device, with the small needle retracted, according to the present disclosure;

FIG. 6 is the top view of the NISE device, with the small needle retracted, according to the present disclosure;

FIG. 7 is the right side view of the NISE device, with the small needle retracted, according to the present disclosure;

FIG. 8 is right side view of the of a NISE device ready or use, according to the present disclosure;

FIG. 9 is the exploded view of the NISE device, according to the present disclosure;

FIG. 10 is a magnified view of the cannula end of the NISE device, according to the present disclosure;

FIG. 11 shows an exemplary top view of an off-the-shelf vascular access device by NxStage modified/converted to the NISE device, according to the present disclosure;

FIG. 12 shows the bottom view of the exemplary off-the-shelf vascular access device by NxStage modified/converted to the NISE device, according to the present disclosure;

FIG. 13 shows the exemplary off-the-shelf vascular access device by NxStage modified/converted to the NISE device with the small needle retracted, according to the present disclosure;

FIG. 14 shows the exemplary off-the-shelf vascular access device by NxStage modified/converted to the NISE device with the small needle and the cannula removed, according to the present disclosure;

FIG. 15 shows the exemplary off-the-shelf vascular access device by NxStage modified/converted to the NISE device ready to begin treatment, according to the present disclosure;

FIG. 16 shows the exploded view of the exemplary off-the-shelf vascular access device by NxStage modified/converted to the NISE device, according to the present disclosure;

FIG. 17 shows a side view of a second embodiment of an off-the-shelf vascular access device by NxStage modified/converted to the NISE device, according to the present disclosure;

FIG. 18 shows the completed assembly of the second embodiment of the exemplary off-the-shelf vascular access device by NxStage modified/converted to the NISE device, according to the present disclosure;

FIG. 19 shows the side view of the completed assembly of the second embodiment of the exemplary off-the-shelf vascular access device by NxStage modified/converted to the NISE device, according to the present disclosure;

FIG. 20 shows the exploded view of the second embodiment of the exemplary off-the-shelf vascular access device by NxStage modified/converted to the NISE device, according to the present disclosure;

FIG. 21 shows various assembled and exploded views of an off-the-shelf vascular access device entitled the Clampcath IV;

FIG. 22 shows an exploded view of an exemplary top view of the off-the-shelf vascular access device Clampcath IV modified/converted to the NISE device, according to the present disclosure;

FIG. 23 shows a top view of the completed assembly of the of the off-the-shelf vascular access device Clampcath IV modified/converted to the NISE device, according to the present disclosure;

FIG. 24 shows a view of the completed assembly of the of the off-the-shelf vascular access device Clampcath IV modified/converted to the NISE device with the small needle retracted, according to the present disclosure;

FIG. 25 shows a side view of the completed assembly of the of the off-the-shelf vascular access device Clampcath IV modified/converted to the NISE device with the small needle retracted, according to the present disclosure;

FIG. 26 shows a view of the completed assembly of the of the off-the-shelf vascular access device Clampcath IV modified/converted to the NISE device with the small needle and NISE assembly retracted, according to the present disclosure;

FIG. 27 shows the view of the off-the-shelf vascular access device Clampcath IV modified/converted to the NISE device after removal of the NISE assembly, according to the present disclosure;

FIG. 28 shows an exemplary alignment jig for cannula assembly with concentric needle stock for use in the NISE device, according to the present disclosure; and

FIG. 29 shows an exemplary alignment jig for assembly with a NxStage device for use in the NISE device, according to the present disclosure

### DETAILED DESCRIPTION OF THE INVENTION

The following detailed description includes references to the accompanying drawings, which forms a part of the detailed description. The drawings show, by way of illustration, specific embodiments in which the invention may be practiced. These embodiments, which are also referred to herein as “examples,” are described in enough detail to enable those skilled in the art to practice the invention. The embodiments may be combined, other embodiments may be utilized, or structural, and logical changes may be made without departing from the scope of the present invention. The following detailed description is, therefore, not to be taken in a limiting sense.

Before the present invention of this disclosure is described in such detail, however, it is to be understood that this invention is not limited to particular variations set forth and may, of course, vary. Various changes may be made to the invention described and equivalents may be substituted without departing from the true spirit and scope of the invention. In addition, many modifications may be made to adapt a particular situation, material, composition of matter, process, process act(s) or step(s), to the objective(s), spirit or scope of the present invention. All such modifications are intended to be within the scope of the disclosure made herein.

Unless otherwise indicated, the words and phrases presented in this document have their ordinary meanings to one of skill in the art. Such ordinary meanings can be obtained by reference to their use in the art and by reference to general and scientific dictionaries.

References in the specification to “one embodiment” indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

The following explanations of certain terms are meant to be illustrative rather than exhaustive. These terms have their ordinary meanings given by usage in the art and in addition include the following explanations.

As used herein, the term “and/or” refers to any one of the items, any combination of the items, or all of the items with which this term is associated.

As used herein, the singular forms “a,” “an,” and “the” include plural reference unless the context clearly dictates otherwise.

As used herein, the terms “include,” “for example,” “such as,” and the like are used illustratively and are not intended to limit the present invention.

As used herein, the terms “preferred” and “preferably” refer to embodiments of the invention that may afford certain benefits, under certain circumstances. However, other embodiments may also be preferred, under the same or other circumstances.

Furthermore, the recitation of one or more preferred embodiments does not imply that other embodiments are not useful and is not intended to exclude other embodiments from the scope of the invention.

As used herein, the terms “front,” “back,” “rear,” “upper,” “lower,” “right,” and “left” in this description are merely used to identify the various elements as they are oriented in the FIGS, with “front,” “back,” and “rear” being relative to the apparatus. These terms are not meant to limit the elements that they describe, as the various elements may be oriented differently in various applications.

As used herein, the term “coupled” means the joining of two members directly or indirectly to one another. Such joining may be stationary in nature or movable in nature. Such joining may be achieved with the two members or the two members and any additional intermediate members being integrally formed as a single unitary body with one another or with the two members or the two members and any additional intermediate members being attached to one another. Such joining may be permanent in nature or alternatively may be removable or releasable in nature. Similarly, coupled can refer to a two member or elements being in communicatively coupled, wherein the two elements may be electronically, through various means, such as a metallic wire, wireless network, optical fiber, or other medium and methods.

It will be understood that, although the terms first, second, etc. may be used herein to describe various elements, these elements should not be limited by these terms. These terms are only used to distinguish one element from another. For example, a first element could be termed a second element, and, similarly, a second element could be termed a first element without departing from the teachings of the disclosure.

Within the specification the following specific definitions or terms may be used:

NISE: Needle Insertion Safer and Easier, an acronym used in this patent application to indicate the devices, their embodiments, methods of manufacture, and methods of use covered under this application.

Vessel: Either an artery or vein, human or animal, natural, graft, artificial, or some combination thereof.

Cutting Edge: A means by which to separate tissue, for example, by incision or blunt dissection, to facilitate entry into a vessel.

Penetrator: A needle, cannula, or similar tubular (but not necessarily circular) structure that includes one or more cutting edges. It may contain various extra holes or other features known to those skilled in the art.

Needle: A penetrator of some kind whose overall shape would be visually recognized as a “needle” by clinicians, even if quite different from typical hypodermic needles.

Small needle: That needle or other penetrator in a NISE device and method for use that first penetrates a vessel.

Cannula: That part of a NISE that enters a vessel after the small needle. It will normally have more than one cutting edge, integrated into a single movable unit, but it may possibly consist of multiple, independently moving parts. (Note: This definition is specific to the NISE; cannula frequently has other meanings elsewhere.) In certain cases, the cannula might be absent from a NISE.

Access needle: A catheter, a hollow needle similar in shape to a typical hypodermic needle, or another penetrator that is typically large relative to the vessel, but is always the final penetrator, and which always remains in the vessel during the treatment or other procedure for which the vessel penetration is made.

Means: A means of doing or implementing an action, feature, and so on, known or obvious to those skilled in the art.

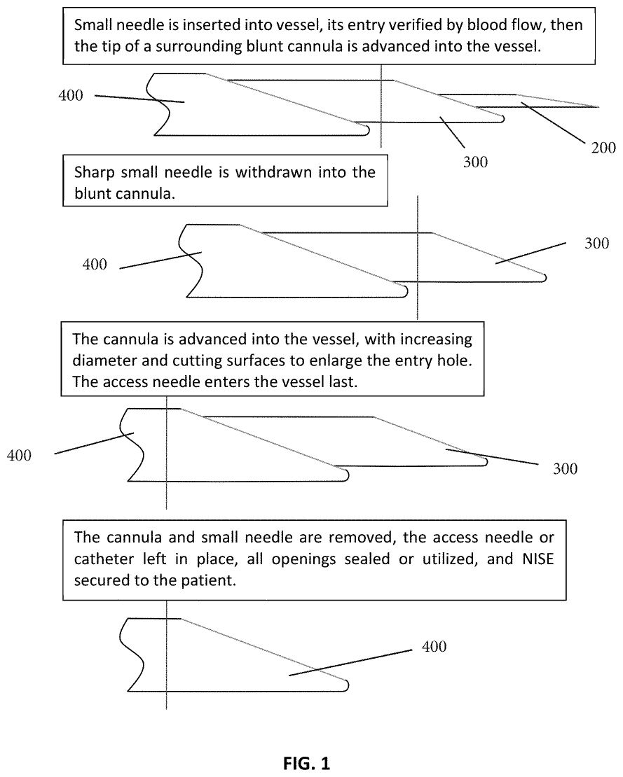

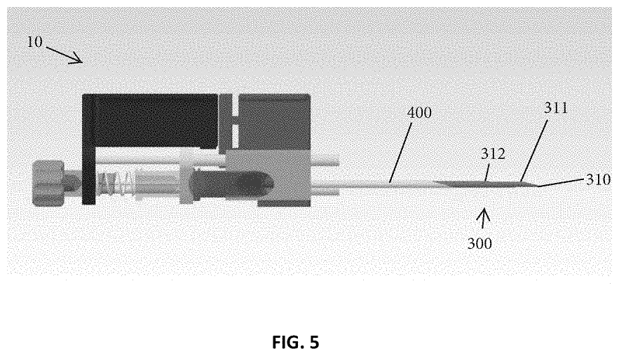

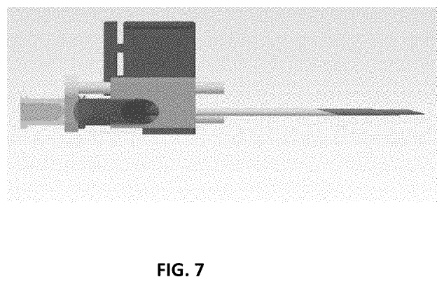

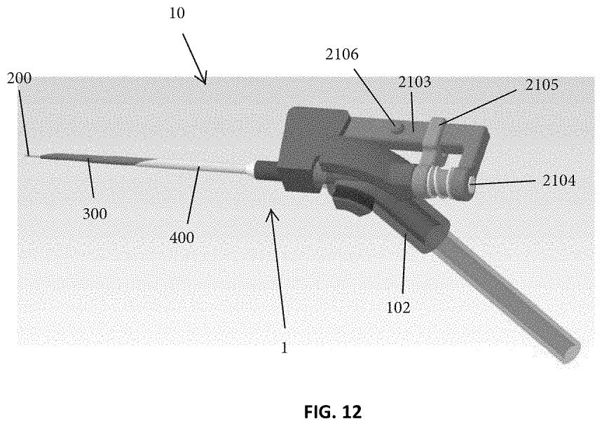

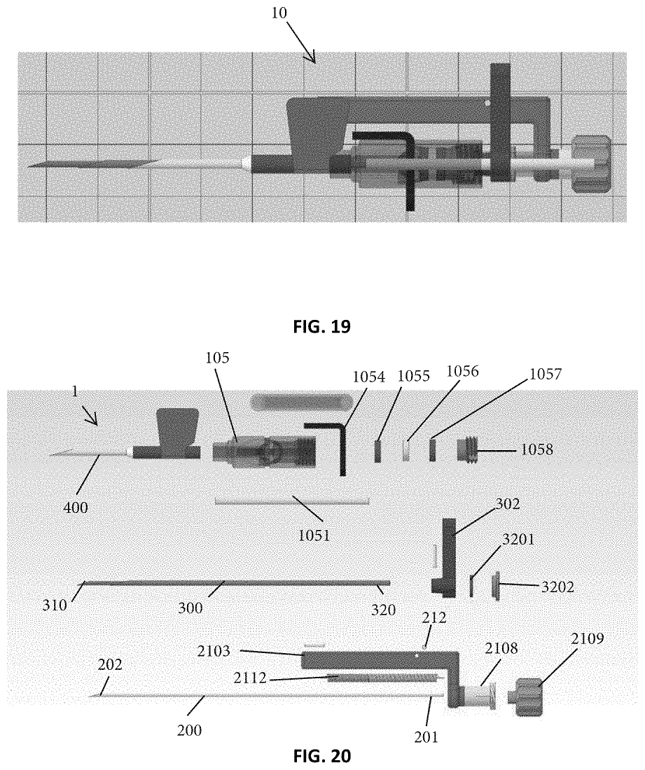

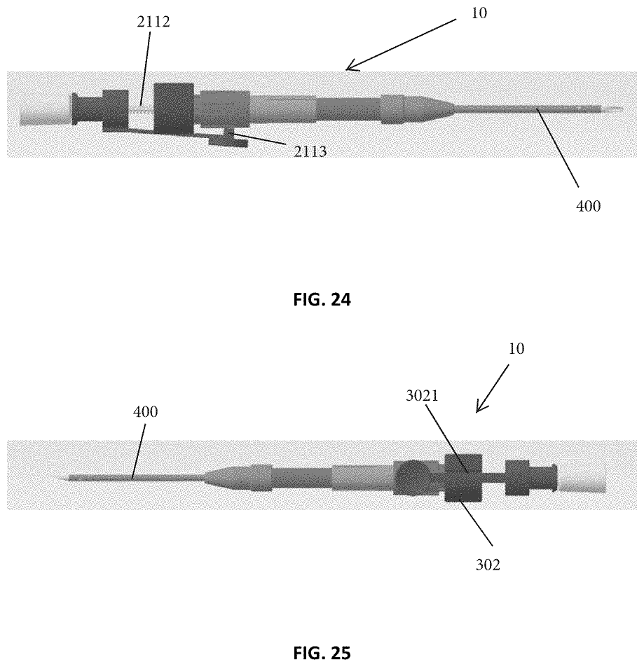

Referring now to FIGS. 1-29, various views of a large needle device configured for insertion into a vessel and a method for use of the device in providing a large needle insertion procedure that is safer and easier. Referring now specifically to FIG. 1 a schematic of the basic stages of a NISE device, referred to herein as device 10, showing insertion within the vessel from top to bottom relative to FIG. 1 is shown. The line represents the insertion site of the vessel wall, without regard to insertion angle or necessary change in insertion angle as insertion progresses. The parts are not to scale with respect to one another or exact relative position. The views are cutaway at the center axis or provided in a side view. First, a sharp penetrator indicated herein as small needle 200 is inserted into the vessel to form an entry hole. Although the schematic depicts normal syringe-needle-shaped points, many penetrator, needle, and needle point shapes are possible as suitable means to first penetrate the vessel, depending on the needs of the specific clinical situation; this applies to all penetrators. Normally, the operator will then verify that the small needle 200 has entered the vessel by the presence of blood emitted or visible from the small needle 200. Normally also, once penetration has been verified, the operator then advances a tip 301 of a surrounding blunt cannula 300 into the vessel, preferably only far enough that resistance to penetration is no longer increasing.

Next the sharp penetrator small needle 200 is retracted while continuing to hold the NISE device 10 in position within the vessel. The retraction may be by manual action, a released spring, or other suitable means. Certain clinical situations may possibly make retraction at other times more appropriate.

The surrounding blunt cannula 300 is then advanced into the vessel all the way (if possible) to enlarge the diameter of the entry hole created by the sharp penetrator small needle 200. This blunt cannula 300 continues penetration through the tip 301. The tip 301 functioning as a penetrator with the tip 301 culminating in a dulled point 310. Although the point 310 of the blunt cannula 300 is dulled, the other surrounding edges 311 are sharp and capable of cutting tissue. Surrounding the cannula 300 is an access needle 400, which is inserted into the vessel last. If the cannula 300 could not be advanced fully during use, then an operator of the device 10 would hold the cannula 300 in position and the access needle 400 is advanced over it and into the target vessel.

When the cannula 300 and access needle 400 are inserted fully into the vessel and the access needle 400 is fully advanced, then the access needle 400 is held in position and the cannula 300 and small needle 200 are removed.

During this injection process a seal (111) between the cannula 300 and access needle 400 (or elsewhere in the access device) prevents or minimizes blood leakage. An additional seal may also be provided between the small needle 200 and the cannula 300. Additional means may also be provided to achieve a complete seal after the cannula 300 and small needle 200 have been removed.

The cannula 300 has both dulled (310) and sharp (311) geometries and a generally enlarging diameter as it passes into the vessel. There are many suitable means to make this penetration and dilation, depending on various clinical situations, which means are known or obvious to those skilled in the art. The cannula 300 may easily be created by stainless steel needle stock in successive layers around the sharp penetrator needle, welded or glued to each other. Although the schematic of FIG. 1 shows that this next stage uses a dulled needle, in actual fact, this and any succeeding stages of the cannula 300 may be constructed of sharp needles. As above, many penetrator shapes, with different dulled and sharp geometries, may be a suitable means to make these create the cannula 300. The NISE device 10 may incorporate a means to secure the NISE device to the patient, such as tape down wings 112 to avoid vessel trauma from movement or the NISE device 10 being dislodged from the vessel. If this means to secure is provided, it should be used just before the time when all interior objects, such as the small needle 200 and cannula 300 are withdrawn and the resultant opening either sealed by suitable means or utilized for blood or other fluid transfer.

As penetration into or within a vessel by a penetrator creates forces in both a vertical and horizontal direction, the hole created within the tissue by the penetrator is often considered to be a shape that can be described as slightly oblong. Therefore, the positional orientation of the NISE device 10 attempts to place any successive cutting portions or points into the largest portion of the initial hole. Accordingly, this NISE device 10 positional orientation enables a smoother advance into the vessel. Because the hole is formed due to the elasticity of the vessel, its shape is independent of prior needle shape or orientation, to a first approximation.

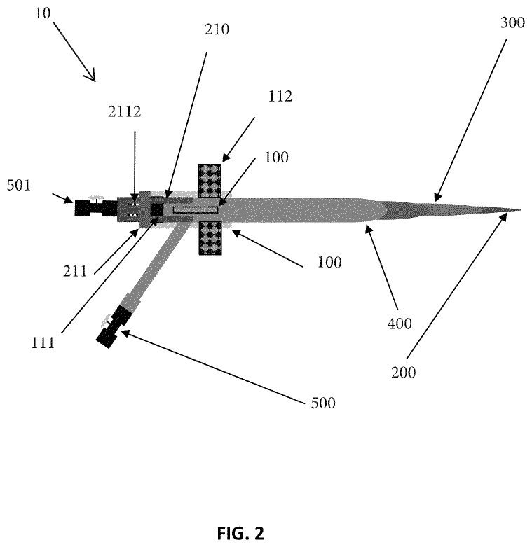

Referring now specifically to FIG. 2, a first embodiment of an exemplary NISE device 10 is shown and generally described in means functionality to disclose the minimal required features and working portions of the device 10. The NISE device 10 at a minimum includes a body portion 100. The body portion 100 generally forming a structure for the working components of the NISE device 10 and generally functions as a chassis, or other means to hold the different parts together and in a proper aligned relationship relative to one another. The body portion 100 may be a stand-alone structure or it may also be integrated into any component of the NISE device 10. One of the more suggested assemblies of the body portion 100 of the NISE device 10 would be to utilize fluid tubing for the main structural components of the body portion 100.

The large access needle 400 is generally the largest needle of the device 10 and is generally positioned as the outermost penetrator of the device 10. The large access needle 400 is generally a hollow structure and functions as a hollow needle or other means to conduct blood or other fluid to or from the patient. If the NISE device 10 is provided without the cannula 300, the large access needle 400 must have a dull tip.

The NISE device 10 includes a port 500. The port 500 being a connection means to conduct fluid from or to the NISE device 10. The port 500 including a means to stop or control fluid flow, such as a valve 501.

Accordingly, the NISE device 10 includes a suitable passageway 101 within the body portion 100 or means for providing a suitable passageway for movement of fluid from the larger access needle 400 to the port 500.

The cannula 300 is positioned internal to the large needle 400 and is normally expected to provide one or more means of continuing to enlarge the hole in the vessel by the presentation of the surrounding edge 311 being a first cutting edge that is followed by a second cutting edge being a successive cutting edge 312 to the vessel, wherein this generally stepped or tapered cannula shape with the surrounding edge 311 and successive cutting edge 312 encouraging expansion of the vessel during insertion and with minimal trauma to surrounding tissues and the vessel.

The small needle 200 or other suitable penetrator means is relatively the smallest diameter penetrator of the NISE device 10 and is required to make the first penetration into the target vessel on a trial basis; the small needle 200 means must cause only minimal trauma if misdirected. The small needle 200 is generally received within an interior space of the cannula 300 in a retractable fashion, wherein the small needle 200 is movable from a position within an interior of the cannula 300 to an exterior position distal to the cannula 300 tip 310 when needed. Accordingly, the cannula 300 has the additional function of providing a protective shield around the small needle 200 until it is advanced to a position exterior the cannula 300 for puncturing the target vessel. In the depicted embodiment (FIG. 2), the small needle 200 is positioned on an actuated portion having flexible wings 210. Accordingly, movement of the small needle 200 is articulated to the flexible wings 210, wherein the flexible wings 210 are movable to lock the small needle 200 into an extended position for penetration and the prevention of rotation of the small needle 200 during use. Release of the flexible wings 210 from the locked position will retract the small needle 200 to a protected position within the cannula 300. Further, the flexible wings 210 along with a fixed handle 211 function as a means to maintain all the needle/penetrators 200, 300, 400 in the correct orientation and coaxial alignment relative to one another and within the relative interior spaces for retraction and extension as needed.

Accordingly, to ensure proper function of the NISE device 10, the small needle 200 is generally received on a means to enable slidable movement, wherein the small needle 200 is movable along the slidable means to advance the small needle 200 past the distal portion of the cannula 300 that follows it and into an extended position for penetrating the target vessel. This means for slidable movement is configured to correspondingly retract the small needle 200 so that it is received completely inside the following cannula 300 so as to protect the vessel from the small needle 200 during additional advancement into the vessel. Preferably this slidable means would be automatic relative to the small needle 200 when the operator directs it, wherein the small needle 200 is retained inside the cannula 300 without further operator attention to it.

Upon proper penetration of the NISE device 10 small needle 200 into the target vessel, a means is provided for the operator to detect blood flow from the vessel into the small needle 200 so as to inform the operator of correct initial placement or otherwise. Preferably, this means is a provided through the valve 501 in communication with the fluid path suitable pathway 101 of the body portion 100.

The NISE device 10 cooperating parts and assembly structures should allow the operator to sense the insertion force of suitable penetrators at all times. If this is not inherent in this preferred configuration, suitable means, such as, but not limited to, a compression, flexible, or resilient member should be provided. In the depicted embodiment, this means to sense insertion force is an elastomeric plug 111. Still further, this suitable means to sense insertion force (111), may also have additional functions to seal blood emission from the small needle 200 to the exterior of the NISE device 10, preferably without operator action.

As fluid control is paramount to the NISE device 10, a means, such as the valve 501 and port 500 is provided to prevent or minimize blood flow to the outside the NISE during placement.

As was mentioned previously in regard to the insertion force, a means is provided to prevent or limit blood or other fluid flow to outside the NISE device 10 specifically during insertion and as the cannula 300 and small needle 200 are removed from the vessel. This means is configured to prevent such flow during the treatment or other procedure, often at considerable pressure (up to 250 mm Hg and −250 mm Hg during dialysis, for example). Specifically, within this FIG. 2 depicted exemplary device, the elastomeric plug 111 is utilized as the means to limit and prevent blood flow to the exterior of the NISE device 10 during use.

During the time that small needle 200 or cannula 300 are being inserted, it is desirable to limit the force against the vessel or other tissue. A means should be provided to do this to prevent either tissue or NISE device 10 damage and allow the operator to assess the situation and take corrective or alternative action. In this preferred exemplary assembly of FIG. 2, this function is provided by defined friction between the cannula outermost surface and the elastomeric plug 111.

To comport with the goals and use of the NISE device 10, all NISE device 10 surfaces in contact with blood or IV fluid or in or near the penetration site must be sterile; where needed, suitable means must be provided to enable needed sterilization. Optionally, a means should be provided so that the NISE device 10 may be successfully inserted with one hand at all insertion stages and be able to be held with one hand while the other hand may be utilized to secure the NISE device 10 to the patient, such as use of the tape down wings 112. Optionally, the above means, features, and concepts may be realized in such fashion that existing off-the-shelf vascular access devices may be incorporated into the NISE with minimal modification to the existing device. Note that the various embodiments of the NISE device 10 and its features depicted schematically in FIG. 2 are not to be construed as the limiting the possible means by which the function of that embodiment may be implemented and thus be covered by this patent. Also, the conceptualized NISE presented schematically in FIG. 2 does not apply completely to any of the embodiments described in paragraphs below.

In another aspect of the present disclosure, the NISE device 10 cannula 300 might not be included, or if included, the number of successive cutting edges 312 (or stages) of the cannula 300 may be other than two. A brief explanation is in order as these variations are within the scope of this disclosure. Clinical needs or situations may arise where a difference between an outer diameter of the small needle 200 and an inner diameter of the access needle 400 may be too small to economically, or even practicably, allow room for the two-stage blunt cannula 300 depicted in FIG. 2. There might only be room for a single-stage blunt cannula 300, or even none at all. The retractable small needle concept is still useful in such a case. Or, on the other hand, a large size difference may even mandate three or more cutting stages in the blunt cannula 300.

In another embodiment of the present disclosure, the NISE device 10 features are incorporated to an existing and already available vascular device by augmenting an off-the-shelf vascular access device by converting it into a NISE device 10.

Accordingly, even simple medical devices and accessories entail high regulatory compliance and approval costs. Once in production, each step of the production process, each part, and the production facility itself, all separately and together entail both high regulatory compliance costs and capital investment. For these same reasons, changes to an existing device or even modifications to production workflow are also expensive. Therefore, it may be desirable to add NISE device 10 components to an existing vascular access device to leverage this existing investment in facilities, training, regulatory, and other costs, while at the same time minimizing changes to the existing device.

The device and method of the present disclosure may be used in combination with an existing vascular access device, wherein the NISE device 10 add-on will advantageously render the existing vascular access device three significant novel features. The basic operating principles resultant in the NISE device 10 when incorporated into existing vascular devices will be virtually the same than that disclosed herein.

Still further, an optional modification to the stock device may be carried out during the incorporation of already available vascular devices: dulling the point of the off-the-shelf device could minimize vessel trauma if the device is improperly inserted or perturbed.

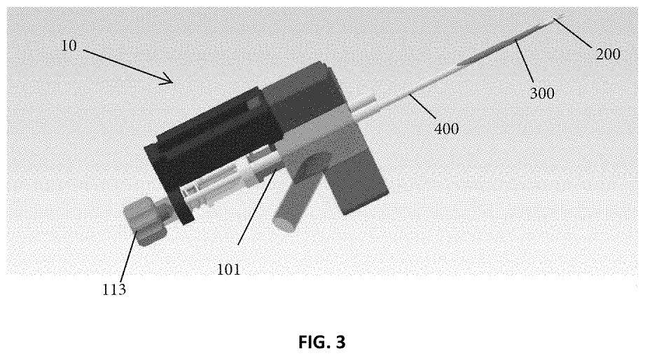

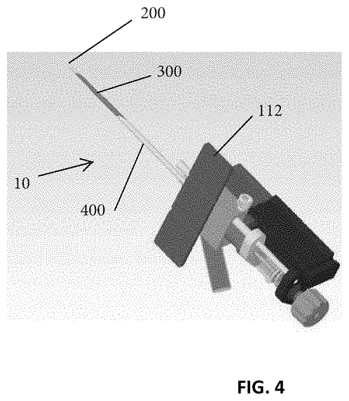

Referring now specifically to FIGS. 3-9, a fully featured and exemplary laboratory prototype NISE device 10 is shown in several views. Although this depicted prototype is aesthetically crude, refinements to both form and function known or obvious to those skilled in the art are expected. An example of such a functional refinement would be to fillet all corners to facilitate thorough cleaning prior to sterilization; this type of refinement does not modify the basic concept. FIGS. 3-4 show the NISE device 10 with the small needle 200 in an extended position beyond the dulled point 310 of the cannula tip 301. In use of the device 10, the operator would first push the small needle 200 forward relative to the device towards the cannula tip 301 and will then hold the two flexible wings 210 so that a pair of protrusions 2101 fit into corresponding slots 2111 in the fixed handle 211. The insertion is then begun by holding the flexible wings 210 in such a manner that the flexible wings 210 are engaged in the corresponding fixed wing slots 2111 of the fixed handle 211. The slots 2111 and protrusions 2101 thus reduce the force the operator needs to exert to retain the sharp penetrator small needle 200 outward and distal to the cannula dulled point 310.

Also, observable are the tape-down wings 112 with which the NISE device 10 may be adhered to the patient and the tube used for fluid withdrawal or infusion (the valve and connector are omitted from the figures). At the far left of the diagram of FIG. 3 and generally referred to as an exit from the passageway 101 is a cap 113, preferably of the leer-lock style, which may be loosened to permit blood flow while inserting the small needle 200, which may then be closed at a convenient time. Alternatively, the cap 113 may contain a hydrophobic membrane filter that will admit gas flow to the outside of the NISE device 10, but not blood or other liquids. The fitting within the passageway 110 into which this cap 113 is coupled is made of a clear or translucent material, so blood is visible through the device 10 without operator interaction. The device 10 may include a supporting means 114 along the axis of the small needle 200, blunt cannula 300, and large needle 400 to keep these structures in proper nested and coaxial alignment. Preferably, this supporting means 114 is a pair of the round tubes/rods on either side of the device 10 to allow for slidable movement of the small needle 200 relative to the blunt cannula 300 and large needle 400 axis to keep everything in alignment, including the direction of the relative points of any penetrating structures.

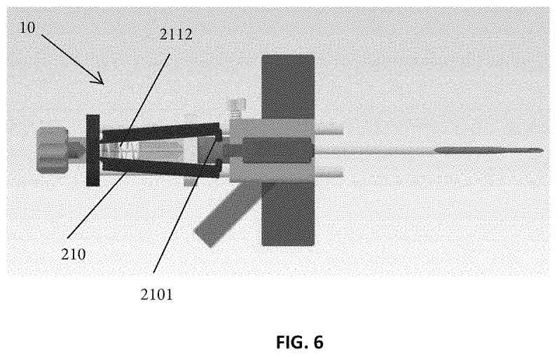

Referring now specifically to FIG. 6, one embodiment of a method by which to allow the flexible wings 210 to either be squeezed to the fixed handle 211 and to spring clear of the fixed handle 211 when released: living hinges in a plastic part. Further, alternate to a living hinge in a plastic part, the device 10 means for retracting the small penetrator 200 may be a spring 2112 with the spring 2112 being a compression spring or general resilient member. Another embodiment could utilize a resilient member formed form a short length of elastomeric tubing.

It is not expected that removal of the small needle by itself will be ordinarily required. In fact, as may be seen in FIG. 6, there is a small tab 2102 on the flexible wings 210 that will lightly strike a nearby plate 115 on the body 100 to preclude the small needle 200 and attached parts from accidentally falling out. However, after the cannula 300 is at least partly inserted into the vessel, the connector 113 that is thus exposed may be used to transfer fluids, a guidewire, and so on, in case a larger lumen of the cannula 300 is needed.

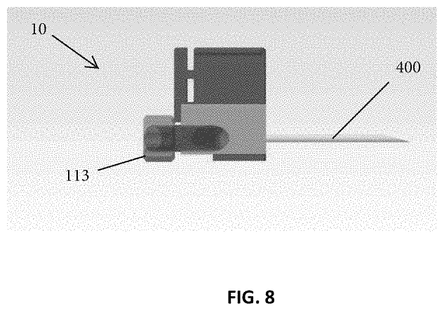

FIG. 8 shows the NISE device 10 ready for tape-down. In this view, the small needle 200 and cannula 300 are removed from the assembly, and an access needle port is capped off though the cap 113.

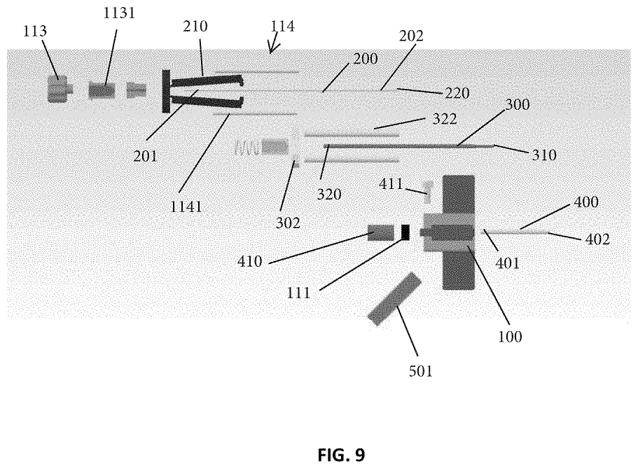

Referring now to FIG. 9, the exploded view of this exemplary prototype is shown to better define and depict the various individual components for this particular exemplary version of the NISE device 10. Referring now to the parts as arranged in orientation relative to the FIG. 9, the top row includes the small needle 200 and parts that move with it during use. The middle row includes the cannula 300 and parts that move with it. The bottom row includes all fixed parts; i.e. parts that are fixed and not considered to move during operation of the device 10 and generally being depicted as fixed to the body 100. Referring now first to the top row and parts related to the movement of the small needle 200 relative to the NISE device 10. The small needle 200 is generally received on the flexible wings 210 and with the small needle 200 extending distal from a fixed end 201 to a free end 202 opposite the fixed end 201 with a distance between the fixed end 201 and the free end 202 defining a length of the small needle 200. The free end 202 generally having a sharpened point 220 for penetrating the target vessel and an internal cavity in fluid communication with the passageway 101, luer cap 113, and luer body 1131 at the fixed end 201. This small needle 200 movable portion may include the supporting means 114 in an alignment rod, tube, or support, generally depicted as a pair of alignment rods 1141.

Moving to the middle row of the parts shown on FIG. 9 related to the cannula 300 starting with the spring 2112 functioning as the means to aid in the retraction of the small needle 200 upon disengagement of the flexible wings 210. The cannula 300 assembly includes the cannula 300 depicted as a two-stage cannula with cutting edges 311 and successive cutting edges 312 and blunt tip 310. Much like the assembly of the small needle 200, the cannula 300 is generally received on a support 302 and with the cannula 300 extending distal from a fixed end 320 to the dulled point 310 opposite the fixed end 320 with a distance between the fixed end 320 and the dulled point 310 defining a length of the cannula 300, with the length of the cannula 300 generally being less than the small needle 200 to enable movement of the small needle 200 exterior to the cannula 300. The dulled point 310 being dull to reduce trauma during vessel insertion and defining an end of an internal cavity in fluid communication with the passageway 101 through a cannula luer 321. The internal cavity of the cannula 300 being coaxial with the small needle 200, wherein the small needle 200 is received within the cannula 300 interior during use. The cannula 300 movable portion may include the supporting means 114 in the form of an alignment rod, tube, or support, generally depicted as a pair of alignment tubes 322. The depicted alignment tubes 322 may be coaxial with and receive the alignment rods 1141 to ensure coaxial movement of the small needle 200 and cannula 300 relative to each other.

Moving on to the bottom row of FIG. 9, related to the fixed or non-movable structures of the NISE device 10, additional device 10 structures are generally fixed to the body portion 100. The body portion 100 including the fixed handle 211 generally providing for the receipt of the flexible wings 210 to secure the small needle 200 in the extended position and coaxial within the large needle 400. Accordingly, the large needle 400 is generally received on the body portion 100 with the large needle 400 extending distal from a fixed end 401 to a free end 402 opposite the fixed end 401 with a distance between the fixed end 401 and the free end 402 defining a length of the large needle 400. The large needle 400 length generally being less than the cannula 300 to enable movement of the cannula 300 to a position exterior the large needle 400. The large needle 400 free end 402 and an internal cavity in fluid communication with the passageway 101 at the fixed end 401. Adjacent the fixed end 401 is the elastomeric seal 111 and a large needle luer 410. The large needle luer 410 providing access to the large needle 400 and associated fluid transfer. The elastomeric seal 111 is designed to cooperate with the large needle luer 410 and generally functioning as a plug to mostly seal against fluid leakage from around the cannula 300 and to provide a defined level of friction against the cannula 300, wherein an excessive amount of insertion force will withdraw the cannula 300. The elastomeric seal 111 and large needle luer 410 may be fastened to the body portion 100 through a fastening means 411, such as, but not limited to a screw 411. Additionally, the body portion 100 provides a suitable connection to interior space or lumen of the large needle 400 for an additional fluid access port 501. This small needle 200 movable portion may include the supporting means 114 in an alignment rod, tube, or support, generally depicted as a pair of alignment rods 1141.

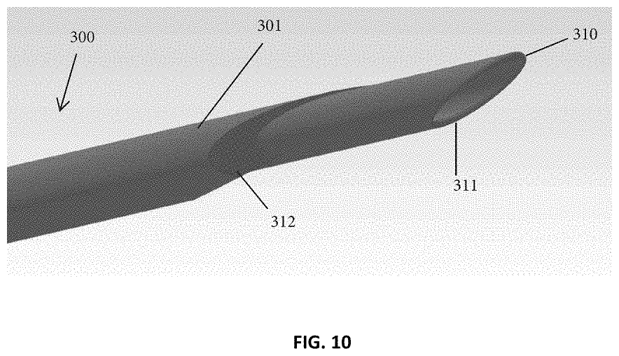

Referring now to FIG. 10, the cannula 300 tip 301 is shown in exploded detail. The cannula tip 301 including the dulled point 310 representing the first edge. This dulled point 310 is utilized to generally spread or widen tissue of the vessel without cutting or trauma. In some applications, this dulled tip 310 may be a desirable feature to further limit possible damage to the vessel. The cannula including a cutting edge 311 along a second stage of the cannula 300 proximal the dulled point 310. These cutting edges 311 are sharp under the assumption that when this cutting edge 311 is advanced, the operator is certain of the position of the penetrator relative to the vessel.

The exemplary shown cannula 300 with two stages (311, 312) is specifically provided in an eccentric alignment. By this eccentric orientation means the leading portion of the second stage 311 is advanced very close to the existing hole in the vessel, facilitating easier insertion of the device 10. The eccentric nature of the cannula 300 results in a structure that is difficult to manufacture within required tolerances. Accordingly, because a multistage cannula 300 allows cuts requiring less force than a single larger penetration at one time, while demanding only one operator interaction with it, it is desired to utilize a manufacturing method that is both feasible for the manufacture of large volumes of accurate and properly sized cannula 300.

Therefore, it has been found in laboratory testing that a less expensive method of manufacturing multistage cannulas 300 is to join successively smaller hypodermic tubing lengths concentrically. Such tubing is readily available in long lengths at reasonable cost, made to precision tolerances, and is available in nesting sizes with diameter differences as low as 51 μm between an inner diameter and the outer diameter of a tube nested inside of it. The various sections are joined together with adhesives, welding, or any method known to those skilled in the art. FIG. 10 shows a close-up of a two stage cannula made by the above method.

There is another feature of the dulled point 310 of the cannula 300 that deserves notice. Most of the cannula tip 301 is at a more or less standard 15 degrees angle and is sharp at the edges. However, the first or leading part of the tip 301 is at a 45 degrees angle to form the slightly dulled tip 310 a tip that is slightly dulled relative to the rest of the cannula tip 301 positioned along the 15 degrees angle. The dulled point 310 is thus still able enter the hole made by the first, sharp penetrator small needle 200 with reasonable force, but is not dangerously sharp within the vessel. In laboratory tests, blood from a calloused fingertip was present after vigorously tapping a cannula tip that was entirely 15 degrees, but slightly dulled. An improved dulled point 310 of 45 degrees where the 45 degrees portion was 12% of the thickness of the cannula 300 measured perpendicular to the axis did not penetrate the author's fingertip. Variations on this method, including more complex geometries, will be obvious by calculation or experimentation to those skilled in the art. Likewise, to meet various clinical needs, other penetrator tip geometries will be obvious or may be derived or empirically determined by those skilled in the art.

In addition to a stand-alone NISE device 10, the safety features and advantages of the assembly can, with some additional structures and materials, be provided as an add-on to existing devices. One such common device is an AV fistula needle with the trademarked name ButtonHole produced by NxStage Medical. This AV fistula needle includes a pair of wings adjacent to a large needle that are normally pinched together and used as handles during insertion of the large needle into the fistula. These wings are then taped down to the patient to secure it from becoming dislodged and prevent the sharp tip from injuring the vessel. The device has a 31 cm tube extending past the wings opposite the large needle that includes, in order, a hanger, a pinch clamp, and a winged leer fitting with cap.

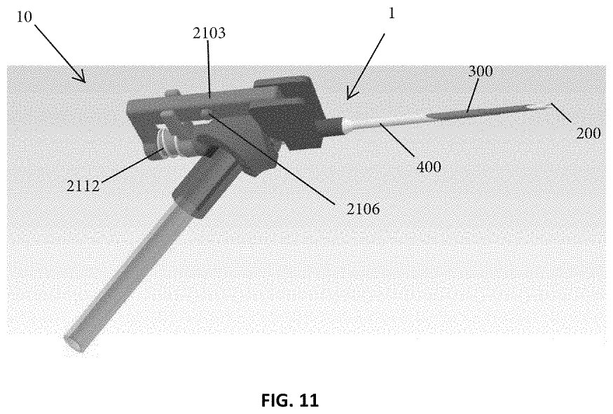

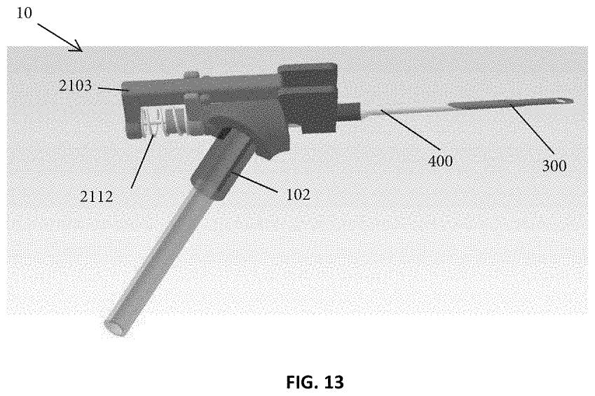

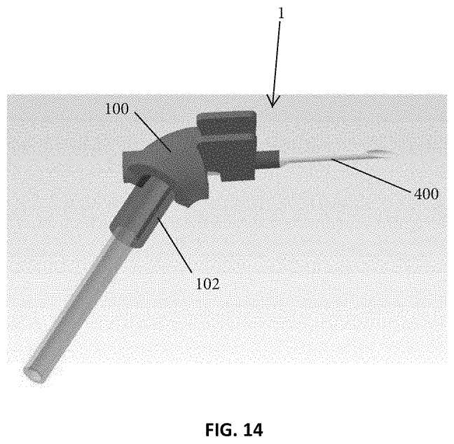

Referring now to FIG. 11-16, a laboratory prototype that demonstrates a first embodiment of how the AV fistula needle by NxStage device 1 may be converted into a NISE device 10. The most immediately visible conceptual difference is that rather that utilize a pair of flexible wings 210 for the movable wings, the movable wing is a single wing 2103, the opposite configuration of the laboratory prototype. There is also no corresponding wing slot 2111 on the fixed handle since the existing wings NxStage device are utilized. But, the principle of the wings/movement is the same; the existing NxStage paired wings are held against the movable wing 2103 and the fingers may be rolled off the portion of the paired wings that pinches the movable wing 2103 to allow the spring 2112 to retract the small needle 200. A bar 2104 and fork 2015 keep the small needle 200 and cannula 300 points aligned. A pair of bumps 2106 (one on each side of the movable wing 2103) provide some resistance to the small needle 200 and movable wing 2103 from falling off accidentally. Other features require more extensive discussion. In FIGS. 13 and 14, the small needle 200 is shown in an extended position.

As shown in FIG. 14, the existing outlet tubing of the NxStage device is modified through the addition of a larger segment of tubing 102 surrounding the exterior of the existing tubing along an outside, with both the large tubing 102 and existing tubing are bent in a smooth 45 degrees arc. The body 100 of this style of NISE device 10 simply clips on to this pair of tubes (102) to hold it in place. This arrangement allows the small needle 200 and cannula 300 to enter an aperture 103 in the body 100 and a position opposite the existing needle of the NxStage device, pierce both the existing tube and the large tubing 102, then enter the existing outer penetrator 400 (access needle). So long as these things are accomplished, the tubing need not be in “perfect” position. An end of the single movable wing 2103 is a hydrophobic membrane 2104 that shows a red dot of blood when blood reaches it from the small needle 200; air can pass the membrane, but not liquid.

The larger segment of tubing 102 provides a more stable and reliable way to hold the add-on components, particularly rotationally, than would be possible using the supplied stock tube. Further, the large tubing 102 provides a defined friction force to allow the inner penetrator pair (small needle 200 and cannula 300) to retract if insertion force becomes excessive, allowing to operator to take appropriate corrective action.

At the completion of insertion, after the small needle 200 and cannula 300 are removed from the body 100 aperture 103, simply straightening the tubes will largely close the hole created by the penetrators. The remaining leak is then fully sealed by simply rotating the outer tube 102 or moving it axially relative to the existing tube. To make this rotation easier, the outer tube 102 may have a square or triangular outer cross-section shape past the body 100 or other easily gripped shape may be desirable to make this easier.

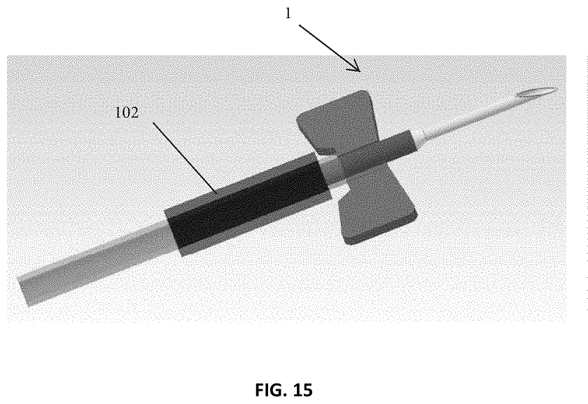

FIGS. 13-15 show the basic stages of inserting the add-on version of the NISE device 10 into a vessel; these stages are the same in concept as described above regarding the stand-alone NISE device 10. FIG. 13 shows the retraction of the small needle 200 after the cannula tip 301 has been inserted. As above, the NISE device 10 is then advanced until the access needle 400 is fully and properly in the vessel. When the NISE device 10 is fully inserted into the vessel it is taped down to help secure it. While holding the NISE device 10 with one hand for further stability, the inner penetrators (small needle 200 and cannula 300) are then removed. The remaining add-on portion of the body 100 is then also removed, and the outer tube 102 is either rotated or moved axially to fully stop any leaks (FIG. 15).

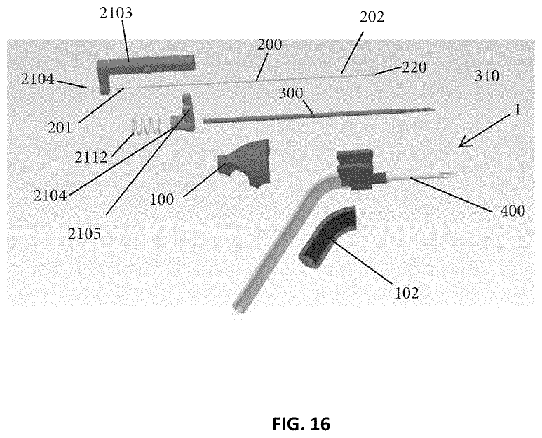

FIG. 16 shows an exploded view of this embodiment of an add-on-version of the NISE device 10 for use with NxStage tubing. It will be understood that some adaptations or modifications may be need for other off-the-shelf products by the same or other manufacturers. Similar to FIG. 9, the exploded view of this exemplary prototype is shown to better define and depict the various individual components for this particular exemplary version of the NISE device 10. Referring now to the parts as arranged in orientation relative to the FIG. 16, the top row includes the small needle 200 and parts that move with it during use. The middle row includes the cannula 300 and parts that move with it. The bottom row includes all fixed parts; i.e. parts that are fixed and not considered to move during operation of the device 10 and generally being depicted as the existing NxStage device along with the large tube 102. Referring now first to the top row and parts related to the movement of the small needle 200 relative to the NISE device 10. The small needle 200 is generally received on the single wing 2103 with the small needle 200 extending distal from the fixed end 201 to the free end 202 opposite the fixed end 201 with the distance between the fixed end 201 and the free end 202 defining the length of the small needle 200. The free end 202 generally having a sharpened point 220 for penetrating the target vessel and in fluid communication with the hydrophobic membrane 2104.

Moving to the middle row of the parts shown on FIG. 16 related to the cannula 300 starting with the spring 2112 functioning as the means to aid in the retraction of the small needle 200 upon disengagement of the single movable wing 2103. Although the spring 2112 is depicted, other means that compress or that are resilient, such as, but not limited to elastomeric tubes or materials, could be utilized. The cannula 300 assembly includes the cannula 300 depicted as a two-stage cannula with cutting edges 311 and successive cutting edges 312 and blunt tip 310. Much like the assembly of the small needle 200, the cannula 300 is generally received on a support, in this case, the supporting structure is the bar 2104 and fork 2105 with the cannula 300 extending distal from the fixed end 320 to the dulled point 310 opposite the fixed end 320 with a distance between the fixed end 320 and the dulled point 310 defining the length of the cannula 300, with the length of the cannula 300 generally being less than the small needle 200 to enable movement of the small needle 200 exterior to the cannula 300. The bar 2104 and fork 2105 additionally function as an alignment means and ensure the small needle 200 and cannula 300 are properly and rotationally aligned. The dulled point 310 being dull to reduce trauma during vessel insertion and defining an end of an internal cavity in fluid communication with the passageway of the existing NxStage device 1 tubing. The internal cavity of the cannula 300 being coaxial with the small needle 200, wherein the small needle 200 is received within the cannula 300 interior during use.

Moving on to the bottom row of FIG. 16, related to the fixed or non-movable structures of the NISE device 10. The NxStage device 1 includes existing tubing, the large needle 400, and wings for grasping and manipulation of the device 1 during use. In this embodiment, the body portion 100 generally includes the aperture 103 at a rear or butt portion of the body portion 100. The aperture 103 generally providing an access to the interior of the body and to the large tube 102 and existing tubing of the NxStage device 1. Accordingly, the movable portions of this embodiment are generally received within this aperture 103. The body portion 100 is generally shaped with an interior channel having an arcuate shape to generally bend and receive the associated tubing (existing tubing and large tube 102) to a relative 45 degrees angle. Wherein, the large tube 102 is placed or slide around the existing tubing, moved into to position generally adjacent the large access needle 400 and existing wing structure and then the body portion 100 is properly positioned on the tubing 102 by aligning the aperture 103 at a position directly opposed the large needle 400 of the existing NxStage device 1. The small needle 200 and cannula 300 can then be placed through the aperture 103 and within in the interior space of the large needle 400 for use.

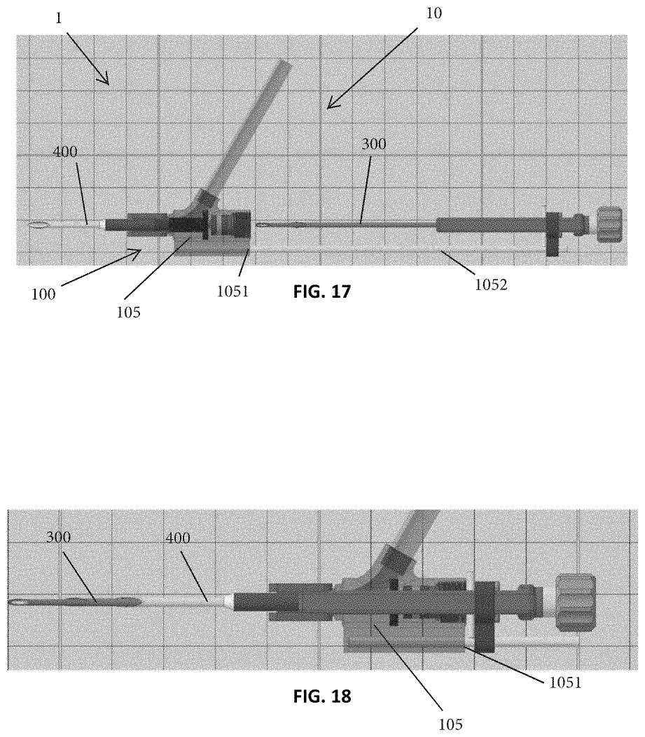

Referring now to FIG. 17-21, a laboratory prototype that demonstrates a second embodiment of how the AV fistula needle NxStage device 1 may be converted into a NISE device 10. In the first embodiment discussed above, the existing tubing of the NxStage device 1 must be punctured to provide an access hole for the small needle 200 and cannula 300 NISE device 10 assembly. This puncture of existing tubing could be problematic for alignment purposes or require modification of existing NxStage tubing with a specifically placed and melted hole for access. Therefore, an additional embodiment of a NISE device 10 configured for attachment to existing NxStage device 1 was developed. This second embodiment does not require a puncture to existing tubing, but rather uses an alternate body 100 shape and features to form a “wye” fitting between the buttonhole needle body (with needle and wings) and the extension set tubing of the existing NxStage device 1.

Referring now to FIGS. 17-20, a second embodiment of a NISE device 10 for attachment to the existing NxStage device 1 shows how a NISE device 10 inner assembly may be accurately loaded into a NxStage device as modified by the addition of a NISE wye 105. The NISE wye 105 generally being a modified body 100 shape for specific fitment with NxStage device 1. During testing, it was found important to insert the NISE device 10 inner assembly straight into the axis of the existing NxStage device 1 large needle 400, and somewhat difficult to do this. For this reason, the NISE wye 105 includes a socket 1051 to accept a length of tube 1052, preferably comprised of stainless steel, functioning similar to the alignment rods 1141 and cannula alignment tubes 322, and a corresponding hole/aperture in the NISE device 10 inner assembly (small needle and cannula) that slides along the tube. If the point of the cannula 310 is in the hole of the NISE wye 105, the proximal end is correctly aligned by the guide tube 1052 so the cannula 300 will go straight into a position nested within the large needle 400. After insertion, the guide tube 1052 may be discarded.

After placement of the NISE wye 105 onto the NxStage device 1, when the wings are pinched together against the small needle 200 assembly, the four penetrator points (small needle 200, dual stage cannula 300, and large needle 400) are essentially aligned together. However, if desired, a shorter alignment tube 1053 may now replace the guide tube 1052; in this penetrator alignment will be better assured and more accurate. A debris protection pin 212 keeps the free end 202 of the small needle 200 just at the end of the cannula 300 during insertion. In the unlikely event that the cannula dulled tip 310 might “core” a silicone septum, the core will appear on the small needle 200; if this should happen, the NISE wye 105 must be discarded unused. The debris protection pin 212 is discarded after insertion of the NISE wye 105 inner assembly into the NISE wye 105. Note that the small needle 200 does not participate in this insertion process, but rather remains inside the cannula 300.

Referring now to FIG. 20, an exploded view of this second embodiment of an add-on-version of the NISE device 10 for use with NxStage tubing. It will be understood that some adaptations or modifications may be needed for other off-the-shelf products by the same or other manufacturers. The exploded view of this exemplary prototype is shown to better define and depict the various individual components for this particular exemplary version of the NISE wye 105 and overall NISE device 10. Referring now to the parts as arranged in orientation relative to the FIG. 20, the top row includes all of the body components 100 related to the NISE wye 105 and all the fixed parts; i.e. parts that are fixed and not considered to move during operation of the device 10 and generally being depicted as the existing NxStage device large needle 400 and exit tubing. The middle row includes the cannula 300 and parts that move with it. The bottom row includes the small needle 200 and the parts that move with it. The parts on the bottom row (small needle) are inserted into the parts in the middle (cannula), and the middle parts are inserted into the parts on the top row (body). Referring now first to the top row and fixed parts relative to the modified NISE device 10. The fixed parts include the existing NxStage device large needle 400, wings, and exit tubing. The NISE wye 105 is configured to couple with the large needle 400 assembly after removal of the existing exit tubing, this exit tubing can then be reinstalled onto the appropriate aperture on the NISE wye 105. To better control fluids, this NISE wye 105 includes several additional sealing elements including, but not limited to, a valve slider 1054, a silicone disk 1055, a spacer ring 1056, a silicone disk with an aperture 1057, and a retainer 1058. The retainer 1058 may be provided with a threaded connection to enable adjustment and compression upon these sealing elements. Additionally, the NISE wye 105 includes the socket 1051 for receipt of the guide tube 1052.

Referring now to the middle row of FIG. 20 and the cannula 300 and associated parts. The cannula 300 is received on the cannula support 302 with the cannula support 302 including an aperture for the receipt of the guide tube 1052 and a pin for retaining a spring of the small needle 200 assembly. The cannula 300 is depicted as a two-stage cannula with cutting edges 311 and successive cutting edges 312 and blunt tip 310. Much like the previous embodiment assemblies, the cannula 300 is generally received on the support 302 with the cannula 300 extending distal from the fixed end 320 to the dulled point 310 opposite the fixed end 320 with a distance between the fixed end 320 and the dulled point 310 defining the length of the cannula 300, with the length of the cannula 300 generally being less than the small needle 200 to enable movement of the small needle 200 exterior to the cannula 300. The cannula fixed end 320 affixed to the support 302 generally includes a silicone disk 3201 and retainer 3202 for the silicone disk 3201. This silicone disk 3201 and retainer 3202 are utilized to seal and prevent leakage between the small needle 200 and cannula 300. Preferably, the silicone disk 3201 is comprised of 10 Shore A silicone material without an aperture. Preferably, the retainer 3202 is provided with a central aperture and glued within the NISE wye 105 body to secure the disk 3201.

Referring now to the bottom row of FIG. 20 and parts related to the movement of the small needle 200 relative to the NISE wye device 105. The small needle 200 is generally received on the single wing 2103, or in this embodiment, the single wing 2103 may more generally be referred to as a holder. The small needle 200 extending distal from the fixed end 201 to the free end 202 opposite the fixed end 201 with the distance between the fixed end 201 and the free end 202 defining the length of the small needle 200. The free end 202 generally having a sharpened point 220 for penetrating the target vessel and in fluid communication with the hydrophobic membrane 2107. In this embodiment, the single wing 2103 or holder includes an integrated luer 2108 with luer cap 2109 for optionally supporting the hydrophobic membrane 2107. The spring 2112 is received on the single wing 2103 and retained for compression through an at least one pin 2109. The debris protection pin 212 is received on the holder 2103 as well.

Another common and existing device or style of device that can be modified to NISE device 10 with some additional structures and materials, is what is commonly referred to as the Clampcath SP 502 or Supercath or SupercathNEO produced by Togo Medikit Co. Ltd. This Clampcath (“CC”) device generally resembles a typical IV catheter with the exception of a flexible translucent portion that can be squeezed during insertion. Although not specifically described herein, it is also likely that the NISE device 10 modifications to the CC product may be incorporated into existing and other IV style catheters without departing from the spirit of this inventive disclosure with the small addition of a translucent area to determine proper vessel entry.

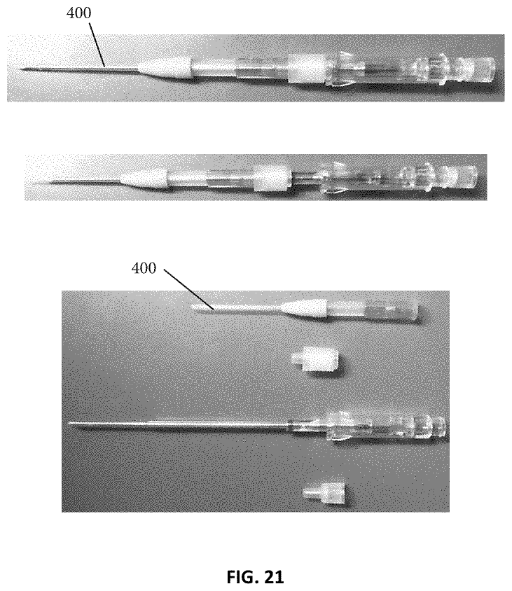

Referring now to FIGS. 21-27, and specifically FIG. 21, an existing CC is shown in an assembled view, a partially separated view, and a view with its major parts separated. A CC device is shown with a CC ready to be inserted into a blood vessel. When it is started into a vessel, an absorbent material in the luer fitting at the extreme right relative to the FIG. 21 will show the red blood. Once the large needle 400 of the CC device is fully inserted, the right-hand half (relative to the FIG.) is withdrawn so that the point is past the clear, flexible tubing that is between the fittings, continuing until the stop is reached. The flexible tubing is then pinched or clamped. The male luer positioned to the right of the female luer adjacent the flexible tubing are then separated. The extension (blood) tubing is then connected to the now exposed female luer and the flexible tubing then unclamped. FIG. 21 middle image shows the two halves partially separated; the clear catheter can be seen at the left side relative to the FIG. 21. FIG. 21 bottom image shows the user separable parts; only the part at the top (large needle 400) remains in the patient during treatment. The male luer, second from the top relative to the placement of parts in FIG. 21 bottom image, contains a soft elastomer seal to contain the blood. The removable part that is third from the top relative to the FIG. 21 bottom image is not used with the NISE device 10, but the bare stainless steel needle is modified and used in the NISE device 10. The blood detection absorbent with its luer is removed from the CC and incorporated into the NISE device 10.

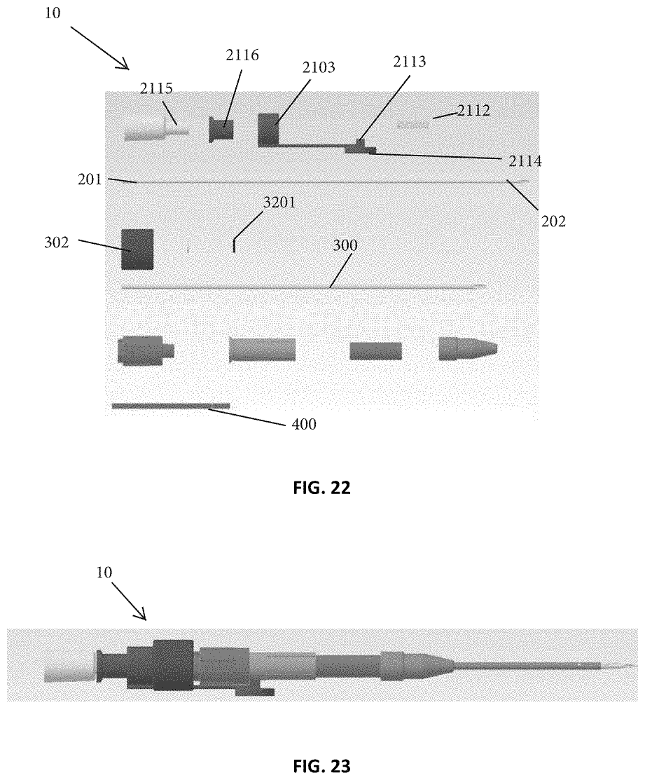

Referring now to FIG. 22, an exploded view of this first embodiment of an add-on-version of the NISE device 10 for use with a CC product. It will be understood that some adaptations or modifications may be needed for other off-the-shelf products by the same or other manufacturers. The exploded view of this exemplary prototype is shown to better define and depict the various individual components for this particular exemplary version of the NISE device 10 when integrated into the existing CC product. Referring now to the parts as arranged in orientation relative to the FIG. 22 the top row includes all of the components related to the small needle 200 that are generally adapted to move within the CC device coaxial to the large needle 400 present in the CC device, the middle row includes the cannula 300 and parts that move with it, the bottom row includes parts that are fixed and not considered to move during operation of the device 10 and generally being depicted as the existing CC device components, including, but not limited to, the CC male luer with seal, the CC female luer and interface to soft tubing, the soft tubing for clamping, the transition luer or element between the soft tubing and the large needle 400 provided on the CC device.

The small needle 200 is generally received on the single wing 2103, or in this embodiment, the single wing 2103 may more generally be referred to as a small needle 200 holder. In this embodiment the small needle holder 2103 includes a retention catch 2113 with the retention catch 2113 generally presenting a raised surface extending perpendicular to a length of the holder 2103 along with a button 2114 to generally retain the small needle 200 in an extended position when desired. The small needle 200 holder 2103 including a male luer 2115 with blood detection absorbent and a female luer 2116 and spring 2112. The small needle 200 extending distal from the fixed end 201 to the free end 202 opposite the fixed end 201 with the distance between the fixed end 201 and the free end 202 defining the length of the small needle 200. The free end 202 generally having a sharpened point 220 for penetrating the target vessel and in fluid communication with the hydrophobic membrane within the male luer 2115.

Referring now to the middle row of FIG. 21 and the cannula 300 and associated parts. The cannula 300 is received on the cannula support 302 with the cannula support 302 including base washer for receipt of the spring 2112 and a small needle 200 to cannula 300 sealing disk 3201. The cannula 300 is depicted as a two-stage cannula with cutting edges 311 and successive cutting edges 312 and blunt tip 310. Much like the previous embodiment assemblies, the cannula 300 is generally received on the support 302 with the cannula 300 extending distal from the fixed end 320 to the dulled point 310 opposite the fixed end 320 with a distance between the fixed end 320 and the dulled point 310 defining the length of the cannula 300, with the length of the cannula 300 generally being less than the small needle 200 to enable movement of the small needle 200 exterior to the cannula 300. The cannula fixed end 320 affixed to the support 302 generally includes a silicone disk 3201. This silicone disk 3201 is utilized to seal and prevent leakage between the small needle 200 and cannula 300. Preferably, the silicone disk 3201 is comprised of 10 Shore A silicone material without an aperture.

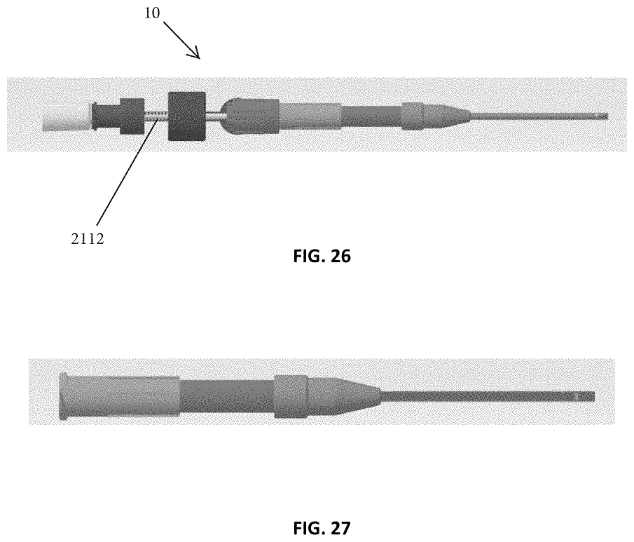

During use of this embodiment, an operator will realize that the small needle holder 2103 has the retention catch 2113 that catches on the CC male luer when the small needle 200 is extended and the button 2114 pressed inward. This action also prevents the small needle 200 or cannula 300 from retracting during initial insertion. When the button 2114 is released, the spring 2112 retracts the small needle 200 into the inside of the cannula 300, as seen in FIG. 22. This immediately protects the blood vessel from its sharp point, and later protects the user from being stuck by the small needle. If the CC/NISE device 10 is then held by the CC female luer, the cannula 300 will be free to retract if an obstruction is encountered (FIG. 26). A slot 3021 in the cannula holder 302 is configured with a shape to receive the small needle 200 holder 2103 to maintain the two needle points (small needle 200 and cannula 300) in alignment and within the large needle 400. Further, within this assembly, the small needle 200 cannot fall out, because the retention catch 2113 will interfere and catch on an edge of the cannula holder 302.

Once the CC/NISE device 10 is placed in the desired position, it may be taped in place near the end. The NISE device 10 portion is then withdrawn until the point of the cannula 300 is entirely past the soft tubing of the CC, but not past the seal in the CC male Luer. The soft tubing is then clamped and the NISE device is entirely withdrawn as seen in FIG. 27. The male luer is also removed. Although the cannula 300 is dull to protect the vessel, if desired, it may be left inside the male luer for additional safety. Extension set tubing is then connected and the soft tubing then unclamped. Additional tape to the patient may then be applied.

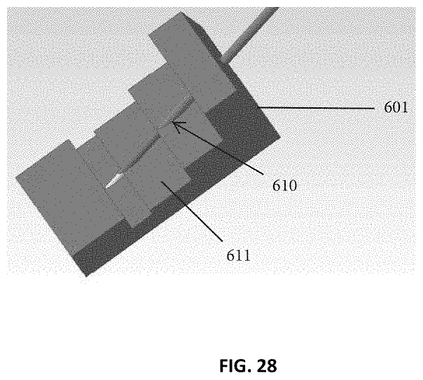

Manufacturing the NISE device 10 has two slightly tricky tasks. If done by precision robots, the manufacturing methods presented below are probably inapplicable. The first method of manufacturing multistage penetrators using concentric nested tubes was described above. However, if using adhesives during assembly, the concentric nested tubes need to be positioned accurately both axially and rotationally, often quickly. In addition, the smallest, sharp penetrator must be the right length relative to the cannula; if it is too long, it cannot be fully retracted and if too short, will not penetrate the vessel properly. Accordingly, a first jig 601, as depicted in FIG. 28 is designed to aid in both of these positioning tasks. Holes 610 are bored to fit each tube diameter, and separate floors 611 are provided for each point to rest upon, which floors 611 have holes 610 that admit the next smaller tube and retain the current tube. The holes 610 and floors 611 thus provide for both axial and rotational alignment.

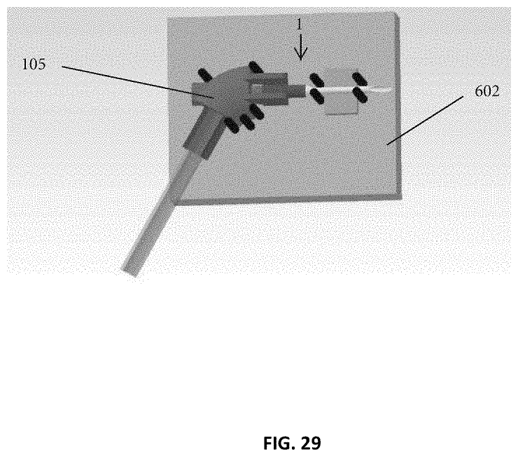

FIG. 29 shows a second jig 602 being a jig that precisely aligns a hole in a butt of an add-on body with the large access needle 400 of an off-the-shelf device, such as, but not limited to the NxStage device previously discussed. The cannula 300 and small needle 200 may be thus reliably inserted in spite of the flexibility of the off-the-shelf device.

For additional NISE device 10 assembly, is also advantageous to make a tool made of a length of sharp pointed tubing that has a hole to receive the end of the smallest cannula tube into its pointed end.

The tool is inserted into the needle end of the host device until it sticks out a bit at the butt end of the body. One may then insert the cannula and small needle or other penetrators together into the tool as it sticks out of the butt of the body, then use the tool to guide the penetrators through everything to final position. This avoids the risk of dulling or damaging the penetrators.

Once the add-on parts and host off-the-shelf device are mated together to form a complete NISE device 10, the completed NISE device 10 must be sterilized. For typical needle assemblies, the fluid path, the needle, and (presumably) the needle cap are sterilized. If either gamma or electron beam irradiation are suitable and utilized, there are no further issues. But there are two potential problem areas if gas is used (e.g., ethylene oxide or steam); the interface between the outer tube and the outlet tube, and the hole created by the cannula.

If the device is merely capped with the access needle cap, sterilant gas can be pumped into the inlet. It will flow through the cap, through and alongside the added penetrators such as the small needle and cannula, then out. That will almost accomplish the sterilization of the fluid path, access needle, and cap. But there will be portions of the wall of the hole around the cannula that will be sealed off against the sterilant due to pressure of that hole wall against the penetrator. Also, when the large tube 102 is rotated or moved axially, its wall would not have been sterilized, so it will result in another small non-sterile place in the fluid path.

If gamma or electron beam sterilization cannot be used, both of these issues must be addressed. With respect to the large and small tubes, one possibility would be to install the outlet port, clamp, and hanger on the outlet tube, and dip the end of the tube in 8% H₂O₂. This concentration of hydrogen peroxide is compatible with PVC and silicone, is not hazardous, and is FDA approved as a liquid sterilant if in the H₂O₂ for six hours. So, a batch of tube ends and large tube segments might be left immersed overnight, as well as a long billed instrument such as a hemostat. The blood tubes are then simply pulled through the small tubes, while still under the H₂O₂. Because the tube tension will squeeze out virtually all of the H₂O₂ (harmless in tiny amounts), the problem surface is sealed off before exposure to atmosphere and water rinsing is not needed. Then the interior of the blood tubes are shaken out, dried, and adhered to the body of the access device as per normal manufacturing of the devices.

As best understood by the time of this disclosure, a completely satisfactory solution to sterilizing the entire surface of the hole created by the penetrator with gas sterilant is lacking. It may be economical to sterilize that small space with a narrow electron beam.

Gas sterilization would then follow. These solutions are admittedly somewhat unwieldy; gamma sterilization would therefore seem to be the best solution for this configuration of the first embodiment of the NISE device 10 incorporated into a NxStage fistula needle.

As noted above, in order to install the outer tube 102, even if the device is gamma-sterilized, the normal manufacturing sequence must be intruded into at the point of attaching the blood tube to the body of the device 10. It would therefore not seem to be all that more intrusive to install a tee or wye at that point, which tee or wye is a modification of the host device, not the add-on system; this is the impetus and reasoning for the development of the second embodiment of the NISE device 10 incorporated with the NxStage device.

While the invention has been described above in terms of specific embodiments, it is to be understood that the invention is not limited to these disclosed embodiments. Upon reading the teachings of this disclosure many modifications and other embodiments of the invention will come to mind of those skilled in the art to which this invention pertains, and which are intended to be and are covered by both this disclosure and the appended claims. It is indeed intended that the scope of the invention should be determined by proper interpretation and construction of the appended claims and their legal equivalents, as understood by those of skill in the art relying upon the disclosure in this specification and the attached drawings.

What is claimed is:

1. A method of needle insertion, the method comprising the steps of:
making large needle insertion into an artery or vein easier and with less unnecessary trauma to the blood vessel.

2. A method as in claim 1, wherein the large needle comprises a first penetrator and a second penetrator, the first penetrator comprising a sharp point and the second penetrator comprising blunted points.

3. A method as in claim 2, wherein the first penetrator has a smaller diameter than the second penetrator.

4. A method as in claim 3, further comprising the steps of:
inserting the first penetrator (sharp needle) into an vessel;
retracting the first penetrator into a cannula;
carrying the second penetrator (access needle) into the vessel;
removing the cannula containing the retracted first penetrator.

5. A method as in claim 4, wherein the cannula has a cylindrical shape and comprises increasing diameters and a cutting outer surface.

6. A method as in claim 4, wherein the cannula comprises a dull tip.

7. A method as in claim 4, wherein the step of removing the cannula includes sealing against leakage.

8. A method as in claim 7, wherein the sealing step includes rotating an outer tube or moving it axially.

9. A device for facilitating large needle insertion into a vessel, the device comprising:
at least two penetrators, a first penetrator of the at least two penetrators comprising a sharp point and a second penetrator of the at least two penetrators comprising blunted points, wherein the first penetrator has a smaller diameter than the second penetrator.

10. A device as in claim 9, further comprising a cannula.

11. A device as in claim 10, wherein the cannula is cylindrical and comprises a dull tip, increasing diameters, and a cutting outer surface.

12. A device as in claim 11, wherein a tip of the cannula surrounds the first penetrator, and the second penetrator surrounds an outer surface of the cannula.

13. A method of manufacturing the device of claim 9.

14. A device to aid in large needle insertion into a vessel, the device comprising:
a body portion; the body portion defining a fluid pathway;
a large needle, the large needle received on the body portion at a fixed position and in communication with the fluid pathway;
a cannula, the cannula coupled to a means to move the cannula coaxially within an interior of the large needle to an extended position at least exterior to an end of the large needle;
a small needle, the small needle coupled to means to move the small needle coaxially within an interior of cannula to an extended position at least exterior to an end of the cannula, wherein the small needle is nestedly received within the cannula and the cannula is nestedly received within the large needle for at least a portion of its movement;
a means to fix the small needle in the extended position;
a means to retract the small needle from the extended position to a protected position within the cannula interior;
a means to seal the small needle, cannula, and large needle from fluid leakage relative to each other and the body portion; and
a port, the port received on the body and in fluid communication with the large needle, the port including a means to stop or control fluid within the fluid pathway.

15. A device as in claim 14, wherein the cannula has a dulled point.

16. A device as in claim 14, wherein the cannula is provided in pair of stages, each stage of the pair of stages including a cutting edge.

17. A device as in claim 15, wherein the cannula is provided in pair of stages, each stage of the pair of stages including a cutting edge.

18. A device as in claim 14, wherein the body portion includes a means to sense the insertion force of the device penetrators.

19. A device as in claim 14, wherein the device includes a means to enable correct orientation of the small needle, cannula, and large needle relative to each other.

20. A device as in claim 14, wherein the device includes a means to detect a flow of blood within the device to ensure proper placement and penetration of the small needle.
